SEQUENCE LISTING (3 sequences)

SEQ ID NO: 1 - protein, 6 aa, Artificial Sequence
  homologous to Thymosin Beta 4
LKKTET

SEQ ID NO: 2 - protein, 7 aa, Artificial Sequence
  homologous to Thymosin Beta 4
KLKKTET

SEQ ID NO: 3 - protein, 7 aa, Artificial Sequence
  homologous to Thymosin Beta 4
LKKTETQ

(19) United States
(12) Patent Application Publication
(10) Pub. No.: US 2009/0053194 A1
(43) Pub. Date: Feb. 26, 2009

(54) METHOD OF TREATING OR PREVENTING BIOLOGICAL OR IMMUNOLOGICAL RESPONSES TO A REACTIVE CHEMICAL OR BIOLOGICAL OR TOXIC AGENT

(75) Inventor: Allan L. Goldstein, Washington, DC (US)

Correspondence Address: ROTHWELL, FIGG, ERNST & MANBECK, P.C., 1425 K STREET, N.W., SUITE 800, WASHINGTON, DC, 20005, US

(73) Assignee: REGENERX BIOPHARMACEUTICALS, INC., Bethesda, MD (US)

(21) Appl. No.: 10/583,852

(22) PCT Filed: Dec. 22, 2004
(86) PCT No.: PCT/US2004/042993

§ 371 Date: Nov. 4, 2008

Related U.S. Application Data
(60) Provisional application No. 60/530,893, filed on Dec. 22, 2003.

(51) Int. Cl. A61K 38/08; A61K 38/16; A61K 39/395; C12Q 1/02; A61P 37/00; A61P 9/00

(52) U.S. Cl. 424/130.1; 514/16; 514/17; 514/12; 424/141.1; 435/29; 424/484

(57) ABSTRACT

A method of treatment for treating, preventing, inhibiting or reducing a biological or immunological response to a reactive chemical agent, biological agent or toxin, by tissue of a subject, includes administering to a subject in need of such treatment an effective amount of a composition including a response-inhibiting agent including amino acid sequence LKKTET [SEQ ID NO: 1], a conservative variant thereof, or an agent that stimulates production of an LKKTET [SEQ ID NO: 1] peptide, or a conservative variant thereof, in the tissue, so as to inhibit the response.

### CROSS-REFERENCE TO RELATED APPLICATION

This application claims the benefit of U.S. Provisional Application Ser. No. 60/530,893, filed Dec. 22, 2003.

### BACKGROUND OF THE INVENTION

1. Field of the Invention

The present invention relates to the field of treating or preventing biological or immunological response to a reactive chemical or biological agent.

2. Description of the Background Art

Contact dermatitis and other allergic reactions due to chemical or biological skin sensitizing agents, environmental toxins or irritants can cause redness, swelling, induration, rashes, blisters, burns, inflammation or eczema skin changes in humans.

Although many treatments have been proposed for such maladies, there remains a need in the art for improved methods and compositions for treating or preventing the erythema, redness, swelling, induration, rashes, itching, blisters and/or inflammation due to the physiological and immunological responses to reactive chemicals, biological agents, or toxins.

### SUMMARY OF THE INVENTION

In accordance with one aspect, a method of treatment for treating, preventing, inhibiting or reducing a biological or immunological response to a reactive chemical agent, biological agent or toxin, by tissue of a subject, comprises administering to a subject in need of such treatment an effective amount of a composition comprising a response-inhibiting agent comprising amino acid sequence LKKTET, a conservative variant thereof, or an agent that stimulates production of an LKKTET peptide, or a conservative variant thereof, in said tissue, so as to inhibit-said response.

### DETAILED DESCRIPTION OF THE INVENTION

Without being found to any specific theory, actin-sequestering peptides such as thymosin beta 4 (Tβ4 or TB4) and other response-inhibiting agents including actin-sequestering peptides or peptide fragments containing amino acid sequence LKKTET or conservative variants thereof, promote reversal or prevention of a biological or immunological response from exposure to a reactive chemical agent, biological agent or toxin. The invention is applicable to conditions including, but not limited to, the following: biological or immunological responses of surface tissues such as skin or mucous membranes, dermatologic and other disorders due to allergic reactions, reactions to chemicals and toxins, contact dermatitis, and reactions to plants including, but not limited to, poison ivy, poison oak, and poison sumac; bites of insects including, but not limited to, mosquitoes, fire ants, chiggers, ticks, bees, spiders, fleas and flies; bites of reptiles, especially venomous reptiles, amphibians, and other animals; contact with various animals with venom on their skin such as poisonous frogs; and allergic reactions of the pulmonary and gastrointestinal systems. The invention is also applicable to skin sensitizing agents, psorasis, atopic dermatitis and eczemas and other conditions that may present with scaling patches and plaques or with bullous and vesicular changes. The invention is also applicable to occupational allergic contact dermatitis, such as but not limited to nickel-associated dermatitis.

Thymosin 4 was initially identified as a protein that is up-regulated during endothelial cell migration and differentiation in vitro. Thymosin 4 was originally isolated from the thymus and is a 43 amino acid, 4.9 kDa ubiquitous polypeptide identified in a variety of tissues. Several roles have been ascribed to this protein including a role in a endothelial cell differentiation and migration, T cell differentiation, actin sequestration, vascularization and wound healing.

In accordance with one embodiment, the invention is a method of treatment for treating, preventing, inhibiting or reducing a biological or immunological response to a reactive chemical agent, biological agent or toxin, by tissue of a subject, comprising administering to a subject in need of such treatment an effective amount of a composition comprising a biological or immunological response-inhibiting agent, which may be a polypeptide comprising amino acid sequence LKKTET, or a conservative variant thereof having biological or immunological response-inhibiting activity, preferably Thymosin β4, and/or Tβ4 isoforms, analogues or derivatives, including KLKKTET, LKKTETQ, oxidized Tβ4, Tβ4 sulfoxide, N-terminal variants of Tβ4, C-terminal variants of Tβ4 and antagonists of Tβ4.

Compositions which may be used in accordance with the present invention include agents such as Thymosin β4 (Tβ4), and/or Tβ4 isoforms, analogues or derivatives, including oxidized Tβ4, Tβ4 sulfoxide, N-terminal variants of Tβ4, C-terminal variants of Tβ4 and antagonists of Tβ4, polypeptides or peptide fragments comprising or consisting essentially of the amino acid sequence LKKTET or conservative variants thereof, having biological or immunological response-inhibiting activity. International Application Serial No. PCT/US99/17282, incorporated herein by reference, discloses isoforms of Tβ4 which may be useful in accordance with the present invention as well as amino acid sequence LKKTET and conservative variants thereof having biological or immunological response-inhibiting activity, which may be utilized with the present invention. International Application Serial No. PCT/GB99/00833 (WO 99/49883), incorporated herein by reference, discloses oxidized Thymosin β4 which may be utilized in accordance with the present invention. Although the present invention is described primarily hereinafter with respect to Tβ4 and Tβ4 isoforms, it is to be understood that the following description is intended to be equally applicable to amino acid sequence LKKTET, peptides and fragments comprising or consisting essentially of LKKTET, conservative variants thereof having biological or immunological response-inhibiting activity, and/or Tβ4 isoforms, analogues or derivatives, including oxidized Tβ4, Tβ4 sulfoxide, N-terminal variants of Tβ4, C-terminal variants of Tβ4 and antagonists of Tβ4.

In one embodiment, the invention provides a method of treatment for treating, preventing, inhibiting or reducing a biological or immunological response to a reactive chemical agent, biological agent or toxin, by tissue of a subject, by contacting the tissue with a biological or immunological response-inhibiting effective amount of a composition which contains a response-inhibiting agent as described herein. As non-limiting examples, the tissue may be selected from a surface tissue such as skin or a mucous membrane of said subject, pulmonary tissue of said subject or gastrointestinal tissue of said subject. The contacting may be topically or systemically. Examples of topical administration include, for example, contacting the skin with a lotion, salve, gel, cream, paste, spray, suspension, dispersion, hydrogel, ointment, or oil comprising a response-inhibiting agent as described herein. Systemic administration includes, for example, intravenous, intraperitoneal, intramuscular injections of a composition containing a response-inhibiting agent as described herein, in a pharmaceutically acceptable carrier such as water for injection.

Response-inhibiting agents for use in the invention, as described herein, may be administered in any suitable biological or immunological response-inhibiting amount. For example, a response-inhibiting agent as described herein may be administered in dosages within the range of about 0.001-1,000,000 micrograms, more preferably in amounts within the range of about 0.1-5,000 micrograms, most preferably within the range of about 1-25 micrograms.

A composition in accordance with the present invention can be administered daily, every other day, etc., with a single application or multiple applications per day of administration, such as applications 2, 3, 4 or more times per day of administration.

Many Tβ4 isoforms have been identified and have about 70%, or about 75%, or about 80% or more homology to the known amino acid sequence of Tβ4. Such isoforms include, for example, Tβ4^{ala}, Tβ9, Tβ10, Tβ11, Tβ12, Tβ13, Tβ14 and Tβ15. Similar to Tβ4, the Tβ10 and Tβ15 isoforms have been shown to sequester actin. Tβ4, Tβ10 and Tβ15, as well as these other isoforms share an amino acid sequence, LKKTET, that appears to be involved in mediating actin sequestration or binding. Although not wishing to be bound to any particular theory, the activity of Tβ4 isoforms may be due, in part, to the ability to polymerize actin. For example, Tβ4 can modulate actin polymerization in skin (e.g. β-thymosins appear to depolymerize F-actin by sequestering free G-actin). Tβ4's ability to modulate actin polymerization may therefore be due to all, or in part, its ability to bind to or sequester actin via the LKKTET sequence. Thus, as with Tβ4, other proteins which bind or sequester actin, or modulate actin polymerization, including Tβ4 isoforms having the amino acid sequence LKKTET, are likely to be effective, alone or in a combination with Tβ4, as set forth herein.

Thus, it is specifically contemplated that known Tβ4 isoforms, such as Tβ4^{ala}, Tβ9, Tβ10, Tβ11, Tβ12, Tβ13, Tβ14 and Tβ15, as well as Tβ4 isoforms not yet identified, will be useful in the methods of the invention. As such Tβ4 isoforms are useful in the methods of the invention, including the methods practiced in a subject. The invention therefore further provides pharmaceutical compositions comprising Tβ4, as well as Tβ4 isoforms Tβ4^{ala}, Tβ9, Tβ10, Tβ11, Tβ12, Tβ13, Tβ14 and Tβ15, and a pharmaceutically acceptable carrier.

In addition, other response-inhibiting agents or proteins having actin sequestering or binding capability, or that can mobilize actin or modulate actin polymerization, as demonstrated in an appropriate sequestering, binding, mobilization or polymerization assay, or identified by the presence of an amino acid sequence that mediates actin binding, such as LKKTET, for example, can similarly be employed in the methods of the invention. Such proteins include gelsolin, vitamin D binding protein (DBP), profilin, cofilin, depactin, Dnasel, vilin, fragmin, severin, capping protein, β-actinin and acumentin, for example. As such methods include those practiced in a subject, the invention further provides pharmaceutical compositions comprising gelsolin, vitamin D binding protein (DBP), profilin, cofilin, depactin, Dnasel, vilin, fragmin, severin, capping protein, β-actinin and acumentin as set forth herein. Thus, the invention includes the use of an EB-inhibiting polypeptide comprising the amino acid sequence LKKTET and conservative variants thereof.

As used herein, the term “conservative variant” or grammatical variations thereof denotes the replacement of an amino acid residue by another, biologically similar residue. Examples of conservative variations include the replacement of a hydrophobic residue such as isoleucine, valine, leucine or methionine for another, the replacement of a polar residue for another, such as the substitution of arginine for lysine, glutamic for aspartic acids, or glutamine for asparagine, and the like.

Tβ4 has been localized to a number of tissue and cell types and thus, agents which stimulate the production of an LKKTET peptide such as Tβ4 or another response-inhibiting agent as described herein, can be added to or comprise a composition to effect production a response-inhibiting agent from a tissue and/or a cell. Such stimulating agents include members of the family of growth factors, such as insulin-like growth factor (IGF-1), platelet derived growth factor (PDGF), epidermal growth factor (EGF), transforming growth factor beta (TGF-β), basic fibroblast growth factor (bFGF), thymosin α1 (Tα1) and vascular endothelial growth factor (VEGF). More preferably, the stimulating agent is transforming growth factor beta (TGF-β) or other members of the TGF-β superfamily. Compositions of the invention may reduce the affects of biological or immunological response to a reactive chemical or biological agent by effectuating growth of the connective tissue through extracellular matrix deposition, cellular migration and vascularization.

In accordance with one embodiment, subjects are treated with a stimulating agent that stimulates production in the subject of a biological or immunological response-inhibiting agent as defined herein.

Additionally, other agents that assist in reduction of biological or immunological response to a reactive chemical agent, biological agent or toxin may be added to a composition along with a response-inhibiting agent as described herein. Such other agents include angiogenic agents, growth factors, agents that direct differentiation of cells, agents that promote migration of cells and agents that stimulate the provision of extracellular matrix material in the tissue. For example, and not by way of limitation, a response-inhibiting agent as described herein alone or in combination can be added in combination with any one or more of the following agents: VEGF, KGF, FGF, PDGF, TGFβ, IGF-1, IGF-2, IL-1, prothymosin α and/or thymosin α1 in an effective amount.

The invention also includes a pharmaceutical composition comprising a therapeutically effective amount of a response-inhibiting agent as described herein in a pharmaceutically acceptable carrier. Such carriers include those listed herein.

The actual dosage or reagent, formulation or composition that provides treatment for treating, preventing, inhibiting or reducing a biological or immunological response to a reactive chemical agent, biological agent or toxin, by tissue of a subject, may depend on many factors, including the size and health of a subject. However, persons of ordinary skill in the art can use teachings describing the methods and techniques for determining clinical dosages as disclosed in PCT/US99/17282, supra, and the references cited therein, to determine the appropriate dosage to use.

Suitable formulations may include a response-inhibiting agent as described herein at a concentration within the range of about 0.001-50% by weight, more preferably within the range of about 0.01-0.1% by weight, most preferably about 0.05% by weight.

The therapeutic approaches described herein involve various routes of administration or delivery of a response-inhibiting agent as described herein, including any conventional administration techniques (for example, but not limited to, topical administration, local injection, inhalation, or systemic administration), to a subject. The methods and compositions using or containing a response-inhibiting agent as described herein may be formulated into pharmaceutical compositions by admixture with pharmaceutically acceptable non-toxic excipients or carriers.

The invention includes use of antibodies which interact with a response-inhibiting agent as described herein. Antibodies which consist essentially of pooled monoclonal antibodies with different epitopic specificities, as well as distinct monoclonal antibody preparations are provided. Monoclonal antibodies are made from antigen containing fragments of the protein by methods well known to those skilled in the art as disclosed in PCT/US99/17282, supra. The term antibody as used in this invention is meant to include monoclonal and polyclonal antibodies.

In yet another embodiment, the invention provides a method of treating a subject by administering an effective amount of stimulating agent which modulates gene expression. The term “modulate” refers to inhibition or suppression of expression when a response-inhibiting agent as described herein is over expressed, and induction of expression when a response-inhibiting agent as described herein is underexpressed. The term “effective amount” means that amount of stimulating agent which is effective in modulating gene expression of a response-inhibiting agent as described herein, resulting in reducing the symptoms of the biological or immunological response to a reactive-chemical agent, biological agent or toxin. A stimulating agent which modulates gene expression of a response-inhibiting agent as described herein may be a polynucleotide, for example. The polynucleotide may be an antisense, a triplex agent, or a ribozyme. For example, an antisense directed to the structural gene region or to the promoter region of a response-inhibiting agent as described herein may be utilized. The stimulating agent which modulates gene expression of a response-inhibiting agent as described herein may also be a small interfering RNAs (siRNAs).

In another embodiment, the invention provides a method for utilizing compounds that modulate activity of a response-inhibiting agent as described herein. Compounds that affect activity of a response-inhibiting agent as described herein (e.g., antagonists and agonists) include peptides, peptidomimetics, polypeptides, chemical compounds, minerals such as zincs, and biological agents.

The invention further relates to a method of screening for a response-inhibiting agent as described herein, comprising contacting a tissue exhibiting a biological or immunological response, with a candidate compound; and measuring a level of reduction of the biological or immunological response in said tissue, wherein a reduction of said level compared to a level in a corresponding tissue lacking said candidate compound indicates that said compound is capable of treating, preventing, inhibiting or reducing the biological or immunological response.

The invention further relates to a method of screening for a response-inhibiting agent as described herein, comprising contacting a tissue with a candidate compound; contacting the tissue with a substance which induces a biological or immunological response in said tissue in the absence of said candidate compound; and measuring a level of reduction of the biological or immunological response in said tissue, wherein a reduction of said level compared to a level in a corresponding tissue lacking said candidate compound indicates that said compound is capable of treating, preventing, inhibiting or reducing the biological or immunological response.

The invention still further relates to a method for screening for a stimulating agent as described herein capable of stimulating production in a tissue of a response-inhibiting agent as described herein, comprising contacting a tissue exhibiting a biological or immunological response as described herein, with a candidate compound; and measuring activity in said tissue of Tβ4 or another response-inhibiting agent as described herein, wherein an increase of activity of Tβ4 or another response-inhibiting agent as described herein, in said tissue, compared to a level of activity of such response-inhibiting agent in a corresponding tissue lacking said candidate compound, indicates that said compound is capable of inducing said stimulating agent.

The invention further relates to a method of screening for a stimulating agent as described herein capable of stimulating production of a response-inhibiting agent as described herein in a tissue, comprising contacting a tissue with a candidate compound, contacting the tissue with a substance that induces a biological or immunological response in said tissue in the absence of said candidate compound; and measuring activity in said tissue of a response-inhibiting agent as described herein, wherein an increase of activity in said tissue of a response-inhibiting agent as described herein, compared to a level of said activity in a corresponding tissue lacking said candidate compound, indicates that said candidate compound is capable of stimulating production in said tissue of a response-inhibiting agent as described herein.

### EXAMPLE

One area of skin surface with a visible redness, induration, swelling and erythema due to exposure to poison ivy was treated by topical application of a medicament containing 2% by weight Tβ4, while another area of visible redness, induration, swelling and erythema due to exposure to poison ivy reaction on the same skin surface was left untreated. After one day, induraton and erythema in the treated area were significantly reduced as compared to the untreated area, and itching of the treated area was significantly less than the untreated area.

1. A method of treatment for treating, preventing, inhibiting or reducing a biological or immunological response to a reactive chemical agent, biological agent or toxin, by tissue of a subject, comprising administering to a subject an effective amount of a composition comprising a response-inhibiting peptide agent comprising amino acid sequence LKKTET [SEQ ID NO: 1] or a conservative variant thereof, Thymosin β4 (Tβ4), a Tβ4 isoform, analogue or derivative, KLKKTET, LKKTETQ, oxidized Tβ4, Tβ4 sulfoxide, an N-terminal variant of Tβ4, a C-terminal variant of Tβ4, Tβ4^{ala}, Tβ9, Tβ10, Tβ11, Tβ12, Tβ13, Tβ14, Tβ15, gelsolin, vitamin D binding protein (DBP) profilin, cofilin, depactin, Dnasel, vilin, fragmin, severinn capping protein, β-actinin, or acumentin, or a stimulating agent that stimulates production of said peptide agent in said tissue, so as to inhibit said response.

2. The method of claim 1 wherein said biological or immunological response comprises redness, induration, swelling, itching, rash, blisters, inflammation, erythema or a combination thereof.

3. The method of claim 1 wherein said response-inhibiting agent has an ability to down-regulate inflammatory cytokines, chemokines or a combination thereof, so as to result in biological or immunological response-inhibition in said tissue.

4. The method of claim 1 wherein said response-inhibiting agent is Thymosin beta 4 (Tβ4).

5. The method of claim 1 wherein said response-inhibiting agent is other than Tβ4.

6. The method of claim 1 wherein said agent comprises amino acid sequence KLKKTET [SEQ ID NO: 2], amino acid sequence LKKTETQ [SEQ ID NO: 3], and N-terminal variant of Tβ4, a C-terminal variant of Tβ4, an isoform of Tβ4, oxidized Tβ4 or Tβ4 sulfoxide.

7. The method of claim 1 wherein said response-inhibiting agent directly and indirectly inhibits said response.

8. The method of claim 1 wherein said response-inhibiting agent indirectly inhibits said response, and said response-inhibiting agent stimulates production of an LKKTET [SEQ ID NO: 1] peptide in tissue of said subject.

9. The method of claim 1 wherein said response-inhibiting agent is administered to said subject at a dosage within a range of about 1-25 micrograms.

10. The method of claim 1 wherein said response-inhibiting agent is administered by direct injection into said tissue, or by intravenous, intraperitoneal, intramuscular, subcutaneous, inhalation, transdermal or oral administration, to said subject.

11. The method of claim 1 wherein said composition is administered systemically.

12. The method of claim 1 wherein said composition is administered topically.

13. The method of claim 12 wherein said composition is in the form of a gel, creme, paste, lotion, spray, suspension, dispersion, salve, hydrogel or ointment formulation, or wherein said peptide agent is present in water.

14. The method of claim 1 wherein said agent is a recombinant or synthetic peptide.

15. The method of claim 1 wherein said agent is an antibody.

16. The method of claim 7 wherein said antibody is polyclonal or monoclonal.

17. The method of claim 1 treatment for treating, preventing, inhibiting or reducing a biological or immunological response to a reactive chemical agent, biological agent or toxin, by tissue of a subject, comprising administering to said subject an effective amount of said composition comprising said stimulating agent that stimulates production of a biological or immunological response-inhibiting polypeptide comprising said peptide agent.

18. The method of claim 17 wherein said polypeptide is Thymosin beta 4.

19. The method of claim 17 wherein said stimulating agent is an agonist of Thymosin beta 4.

20. The method of claim 1, wherein said tissue is a surface tissue selected from skin or a mucous membrane of said subject, pulmonary tissue of said subject or gastrointestinal tissue of said subject.

21. The method of claim 17, wherein said tissue comprises a surface tissue selected from skin or a mucous membrane of said subject, pulmonary tissue of said subject or gastrointestinal tissue of said subject.

22. A method of screening for a biological or immunological response-inhibiting agent, comprising contacting tissue exhibiting a biological or immunological response, with a candidate compound; and measuring a level of reduction of the biological or immunological response in said tissue, wherein a reduction of said level compared to a level in a corresponding tissue lacking said candidate compound indicates that said candidate compound is capable of treating, preventing, inhibiting or reducing said biological or immunological response.

23. A method of screening for a biological or immunological response-inhibiting agent, comprising contacting tissue with a candidate compound; contacting the tissue with a substance which induces a biological or immunological response in said tissue in the absence of said candidate compound; and measuring a level of reduction of the biological or immunological response in said tissue, wherein a reduction of said level compared to a level in a corresponding tissue lacking said candidate compound indicates that said compound is capable of treating, preventing, inhibiting or reducing the biological or immunological response.

24. A method for screening for a stimulating agent capable of stimulating production in a tissue of a biological or immunological response-inhibiting agent, comprising contacting a tissue exhibiting a biological or immunological response, with a candidate compound; and measuring activity in said tissue of a biological or immunological response-inhibiting agent, wherein an increase of activity of said response-inhibiting agent in said tissue, compared to a level of activity of said response-inhibiting agent in a corresponding tissue lacking said candidate compound, indicates that said compound is capable of inducing said stimulating agent.

25. The method of claim 24 wherein said response-inhibiting agent is an LKKTET [SEQ ID NO: 1] peptide.

26. The method of claim 25 wherein said LKKTET [SEQ ID NO: 1] peptide is Thymosin beta 4.

27. A method of screening for a stimulating agent capable of stimulating production of a biological or immunological response-inhibiting agent in a tissue, comprising contacting a tissue with a candidate compound, contacting the tissue with a substance that induces a biological or immunological response in said tissue in the absence of said candidate compound; and measuring activity in said tissue of said response-inhibiting agent, wherein an increase of activity in said tissue of said response-inhibiting agent, compared to a level of said activity in a corresponding tissue lacking said candidate compound, indicates that said candidate compound is capable of stimulating production in said tissue of said response-inhibiting agent.

28. The method of claim 27 wherein said response-inhibiting agent is an LKKTET [SEQ ID NO: 1] peptide.

29. The method of claim 28 wherein said LKKTET [SEQ ID NO: 1] peptide is Thymosin beta 4.
